(19) United States
(12) Patent Application Publication
(10) Pub. No.: US 2023/0226343 A1
(43) Pub. Date: Jul. 20, 2023

(54) INFLATABLE MEDICAL DEVICES, METHODS OF MANUFACTURE AND USE

(71) Applicant: SHIFAMED HOLDINGS, LLC, Campbell, CA (US)

(72) Inventors: Tom SAUL, Portland, OR (US); Amr SALAHIEH, Saratoga, CA (US)

(21) Appl. No.: 17/998,614

(22) PCT Filed: May 12, 2021
(86) PCT No.: PCT/US2021/031993

§ 371 Date: Nov. 11, 2022

Related U.S. Application Data
(60) Provisional application No. 63/023,730, filed on May 12, 2020.

(51) Int. Cl. A61M 60/205

(52) U.S. Cl. CPC A61M 60/205; A61F 2250/0003

(57) ABSTRACT

Intravascular inflatable medical devices and their methods of manufacture and use. The inflatable medical devices may include a conduit that includes an inflatable wall, with the inflatable wall defining a lumen therein. The inflatable wall may include an outer layer and an inner layer, and optionally an intermediate layer between the inner and layers. Intermediate layers may include one or more couplings between the outer and inner layers, and may include radial connectors extending between the outer layer and the inner layer.

### CROSS REFERENCE TO RELATED APPLICATIONS

This application claims priority to U.S. App. No. 63/023,730, filed May 12, 2020, the entire disclosure of which is incorporated by reference herein for all purposes.

### INCORPORATION BY REFERENCE

The disclosures of the following publications are incorporated by reference herein in their entireties for all purposes: WO2018/226991, WO2019/094963, WO2019/152875, and WO2019/152875.

All publications and patent applications mentioned in this specification are herein incorporated by reference to the same extent as if each individual publication or patent application was specifically and individually indicated to be incorporated by reference.

### BACKGROUND

There are a variety of medical devices that are adapted to expand within some region of the anatomy, and when expanded define or create a lumen passing therethrough. The medical devices may include devices that are intended to be implanted, used temporarily and then removed, as well as delivery devices that may be used to delivery and position a medical device or tool, such as delivery catheter or sheaths. Examples includes expandable stents, expandable grafts, intravascular blood pumps that include a fluid conduit through which blood is moved, gastrointestinal or pulmonary devices for maintaining patency in lumens within tumors as used in cancer treatments, etc.

Regardless of the application, it may be beneficial to have additional types of expandable devices that may be used.

### SUMMARY OF THE DISCLOSURE

One aspect of the disclosure is related to medical devices that include an inflatable wall.

In this aspect, the inflatable wall may include a radially outermost surface and a radially innermost surface, and an intermediate layer or region disposed between the outermost surface and the innermost surface.

In this aspect, the inflatable wall may include an outer layer or member and an inner layer or member.

In this aspect, the inflatable wall may include an intermediate layer that is disposed radially between the outer layer and the inner layer, wherein the inner layer may at least partially define an inner lumen.

In this aspect, the intermediate layer may include, in a cross-section transverse to a longitudinal or long axis of the inflatable wall when inflated, an inflatable volume and a plurality of radial connectors extending between the outer layer and the inner layer. The radial connectors may be arranged and configured such that when a fluid is delivered into the inflatable volume, a stiffness of the inflatable wall increases.

In this aspect, an inflatable wall may be compliant when uninflated such that it is adapted to be folded for delivery, wherein when folded it can have a different general configuration than when inflated. For example, when inflated it may assume a cylindrical configuration, but it may be adapted to be bunched or folded up with a random or non-defined shape or configuration that is not cylindrical.

In this aspect, a plurality of radial connectors may be arranged so as to have a regular pattern along a length of an intermediate layer.

In this aspect, a plurality of radial connectors may be arranged so as to have an irregular pattern along a length of an intermediate layer.

In this aspect, a plurality of radial connectors may be arranged within an intermediate layer such that a first portion of the inflatable wall has a first stiffness and a second portion spaced from the first portion has a second stiffness that is different than the first stiffness when the inflatable wall is inflated with a fluid.

In this aspect, the device may further comprise a fluid pathway extending, such as extending proximally, from the inflatable wall and in fluid communication therewith to facilitate fluid delivery through the fluid pathway and into the inflatable wall.

In this aspect, each of the radial connectors may extend from an outer layer at a location that is one or more of axially or circumferentially spaced from a location from which the respective radial connector extends from an inner member. A plurality of radial connectors may together create a wave pattern.

In this aspect, the inflatable volume may comprise a first inflatable volume and a second inflatable volume, wherein the first and second inflatable volumes may not be in direct fluid communication within the inflatable wall. First and second inflatable volumes may be in fluid communication with first and second fluid inflation pathways, respectively.

In this aspect, the device may include an intermediate layer that includes, in a cross-section transverse to a long axis of the inflatable wall when inflated, an inflatable volume and a plurality of radial connectors extending between the outer layer and the inner layer. A plurality of radial connectors may each have a length from a first end that is coupled to an outer layer to a second end that is coupled to an inner layer. Lengths of a plurality of radial connectors may be greater than a radial thickness of the outer layer and a radial thickness of the inner layer, the radial thickness measured in a radial direction transverse to the long axis of the inflatable wall.

One aspect of the disclosure is an intravascular inflatable medical device that includes a conduit including an inflatable wall, the inflatable wall defining a lumen therein. The inflatable wall may include, in a cross-section transverse to a long axis of the inflatable wall when inflated, an inner layer and an outer layer secured to the inner layer at a plurality of circumferentially spaced secured locations.

In this aspect, and in the cross section, the inner layer may have an inner length between first and second circumferentially spaced secured locations that is greater than an outer layer length between the first and second circumferentially spaced secured locations, such as shown in FIGS. 15A and 15B, for example without limitation.

In this aspect, the inner layer, in the cross section and in between adjacent circumferentially spaced secured locations, may partially define a plurality of circumferentially adjacent fluid pathways (e.g., such as shown in FIG. 15B), and wherein each of the circumferentially adjacent fluid pathways is configured to interfere with at least one adjacent fluid pathway when inflated with a fluid to thereby stiffen the inflatable wall.

In this aspect, the inner member may have a corrugated pattern comprising a plurality of ridges and a plurality of grooves when the inflatable wall is inflated. The inner member may be coupled to the outer member at the plurality of grooves and not at the plurality of ridges, wherein the plurality of ridges face radially inward towards the long axis.

In this aspect, the inflatable wall may comprise a plurality of axially extending fluid pathways that are parallel with a longitudinal or long axis of the inflatable wall when inflated.

In this aspect, the inner member may have a wave pattern in the cross section when the inflatable wall is inflated, the wave pattern comprising a plurality of peaks and a plurality of valleys, wherein the plurality of peaks face radially inward towards the long axis.

In this aspect, the intermediate layer may comprise a plurality of axially extending fluid pathways that are parallel with a long axis of the conduit when inflated. A plurality of axially extending fluid pathways may have a closed distal end. At least one of a plurality of axially extending fluid pathways has an open distal end that defines a fluid exit port, such as in the example shown in FIG. 18.

One aspect of the disclosure includes methods of manufacturing any of the inflatable walls herein. Methods of manufacturing may include coupling an inner layer to an outer layer, and optionally coupling an intermediate layer to the outer layer and to the inner layer.

### BRIEF DESCRIPTION OF THE DRAWINGS

FIG. 1 illustrates an exemplary conduit comprising an inflatable wall.

FIG. 2A illustrates an exemplary conduit comprising an inflatable wall.

FIG. 2B illustrates an exemplary inflatable wall in an uninflated state.

FIG. 2C illustrates an exemplary inflatable wall in an inflated state.

FIG. 3A illustrates a sectional view of an exemplary inflatable wall in an inflated state.

FIG. 3B illustrates a portion of the sectional view of the exemplary inflatable wall from FIG. 3A.

FIG. 4 illustrates an exemplary inflatable wall including exemplary radial connectors.

FIG. 5A illustrates exemplary components that may be used in the manufacture of an inflatable wall.

FIG. 5B illustrates an exemplary transverse section of an exemplary inflatable wall.

FIG. 5C illustrates an exemplary transverse section of an exemplary inflatable wall.

FIG. 6 illustrates an exemplary tie layer or tie member.

FIG. 7 illustrates an exemplary intermediate member in a flattened configuration.

FIG. 8 illustrates an exemplary transverse sectional view of an exemplary inflatable wall in an inflated state.

FIG. 9 illustrates an exemplary transverse sectional view of an exemplary inflatable wall in an inflated state.

FIGS. 10A and 10B illustrate sectional views of an exemplary inflatable wall including a plurality of radial connectors.

FIGS. 11A and 11B illustrate an exemplary implementation of an inflatable wall in an exemplary intravascular blood pump.

FIG. 12 illustrates an exemplary implementation of an inflatable wall in an exemplary intravascular blood pump.

FIG. 13 illustrates an exemplary implementation of an inflatable wall in an exemplary intravascular blood pump.

FIGS. 14A, 14B, 14C, and 14D illustrate an exemplary implementation of an inflatable wall.

FIGS. 15A and 15B illustrate an exemplary conduit having an inflatable wall.

FIGS. 16A, 16B and 16C illustrate an exemplary method of manufacturing an exemplary inflatable wall.

FIGS. 17A, 17B and 17C illustrate an exemplary conduit having an inflatable wall.

FIG. 18 illustrates an exemplary implementation of an inflatable wall in a medical device.

### DETAILED DESCRIPTION

The disclosure is related to medical devices that include an inflatable wall. The devices may include a conduit that includes the inflatable wall, which may be used or adapted for use in a variety of medical applications, and may be referred to herein generally as inflatables. The exemplary devices and uses herein are intended to be illustrative and not intended to be limiting. The disclosure provides examples of inflatable walls that are adapted to be inflated with a fluid, wherein fluid herein includes gases and liquids. The devices can be used in a variety of applications, including applications for which an expandable component is desired. When inflated, the inflatable wall generally defines a lumen passing therethrough. The inflatable wall may be used as an implant or one or more components thereof, as part of temporarily positioned devices, and/or components of a delivery system. An exemplary advantage of inflatables herein is that when they are not inflated, the structures are flexible and compliant and can be easily deformed and collapsed into a smaller cross profile for delivery, and when they are inflated, their stiffness increases significantly and the inflatable assumes or moves towards a deployed or operational configuration. (e.g., generally cylindrical). While the inflatable structures are compliant, the materials from which the inflatable structures are comprised may be relatively stiff. For example, a component may be relatively stiff but may also be relatively thin-walled. These properties can be highly advantageous when it's needed to collapse/deform a medical device to a smaller profile to deliver it to a target location, then expand it to assume a greater stiffness for use.

While some known medical devices (e.g., stents) are rather constrained in the manner in which they can collapse due to their materials and construction, inflatables herein offer a significant advantage in that they are not as limited in the manner in which they collapse, and do not necessarily have a particular collapsed configuration that they will tend to assume as would some traditional expandable structures. They can be collapsed easily, but may not be constrained to collapse predictably into a particular configuration, but rather may assume a wide variety of deformed/collapsed states which all facilitate collapsed delivery. The inflatables herein may be thought of as being adapted to fold, bunch up, or scrunch up, when collapsed, which is in contrast to typical stents, for example, which have collapsed configurations that are generally similar to the expanded configurations, but with a reduced diameter. This characteristic can allow the inflatables to be much more easily and less regularly collapsed. This may provide significant advantages for delivery, as the inflatables can occupy less space/volume within a delivery device than traditional devices, which generally deform in a more predictable and limited manner. The inflatables herein can be thought of to some extent as being able to assume the shape of the volume in which they are placed, rather than having a particular and limited collapsed configuration. This may provide more design flexibility because the constraints around delivery and the delivery configuration may be relaxed. Additionally, as is set forth herein, the inflatables can also be easily inflated for use, after which their stiffness increases significantly, offering the rigidity and support needed for a wide variety of medical applications.

An exemplary but not-limiting implementation of an inflatable wall is as an inflatable cylindrical structure that defines an internal lumen. One merely exemplary and non-limiting use of the inflatable walls herein is for intravascular blood pumps, wherein fluid may be moved through the inflatable wall.

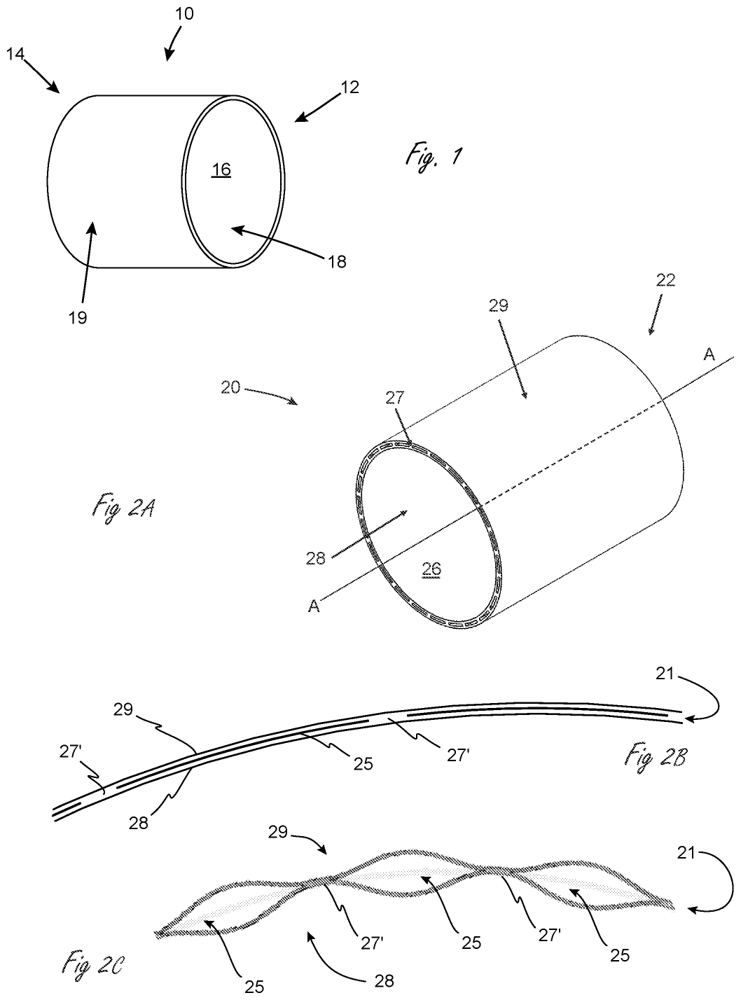

FIG. 1 illustrates an exemplary conduit, which is this example and other examples herein includes an inflatable wall, as shown. Conduits described herein are understood to include an inflatable wall (such as shown in FIG. 1) and may be described as such, wherein the inflatable wall defines a lumen therein (such as lumen 16). In this example, the conduit includes an inflatable wall 10 that has a cylindrical configuration when inflated, as shown. The conduit and inflatable wall 10 include a first end 12 and a second end 14. An inner surface 18 of the inflatable wall 10 defines a lumen 16. The conduit and inflatable wall 10 include a radially outer surface 19. Any aspect of inflatable wall 10 may be incorporated with or into any other inflatable walls or conduits herein, and vice versa.

FIGS. 2A, 2B and 2C illustrate an exemplary conduit including an inflatable wall 20. Wall 20 includes first end 22 and a second end not shown in this sectional view, outermost radial surface 29 (which may comprise more than one individual discrete surface of material), and innermost radial surface 28 defining lumen 26 therein. Wall 20 also includes intermediate layer 21, which in this example includes one or more radial connects 27 and inner volume 25. “Intermediate layer” (or similar derivatives thereof) as used in this context and in any of the embodiments herein does not necessarily impart a particular structural construction, but can generally refer to a region of the inflatable wall in between a radially outermost surface (e.g., surface 29) and a radially innermost surface (e.g., surface 28). An intermediate layer does not necessary imply an entire region between radially outermost and innermost surfaces, but may refer to a region therein. For example, in some embodiments, an intermediate layer may include radial connects that include locations where an inner member and an outer member are secured together (e.g., welded).

In the sectional views of FIGS. 2B and 2C (uninflated (but not collapsed) and inflated, respectively), the inner volume appears to include individual pockets 25, but in any of the embodiments herein, the inflatable wall may have an inner inflatable volume (e.g., volume 25) that is continuous along any length of the conduit, including the entire length or substantially the entire length of the conduit. In FIGS. 2B and 2C, for example, all pocket regions of volume 25 may thus be in fluid communication with each other (described in more detail below). Radial connects 27′ in FIGS. 2B and 2C have minimal thickness as would be the case if the inner and outer members were welded together at these locations, in contrast to those illustrated in FIG. 2A, where the radial connects comprise a larger radial dimension associated with radial connects 27.

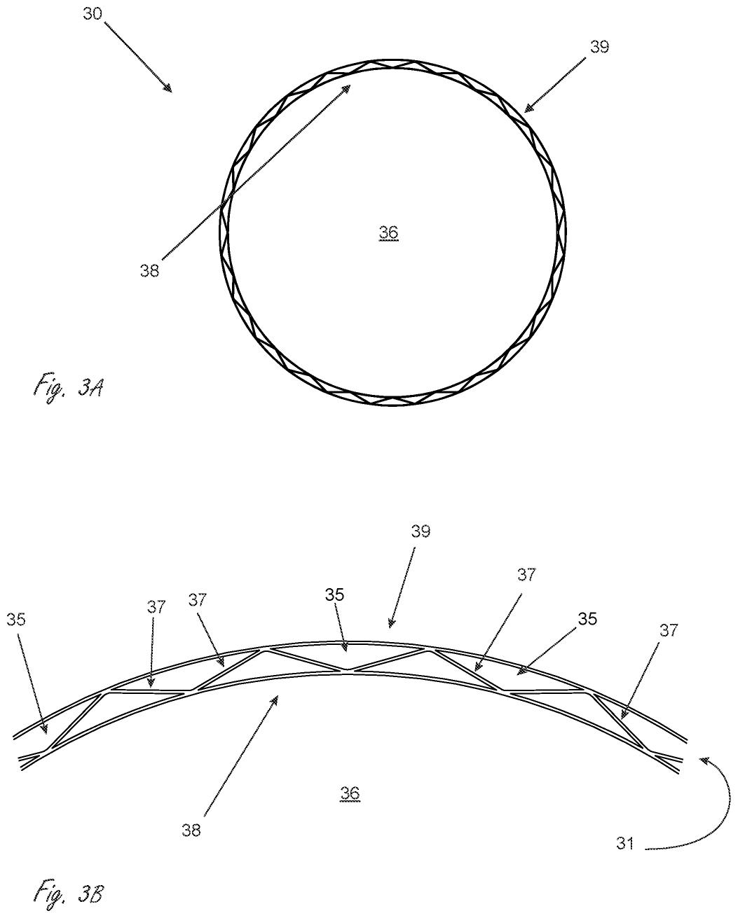

As used herein, “radial connect” or other derivative thereof (e.g., “radial connector”) refers to one or more structural components that alone or together have some radial dimension. That phrase does not impart a limitation that the radial connector extends solely or primarily in the radial direction transverse to a long axis of the inflatable wall when inflated (such as axis A-A in FIG. 2A). While connectors 27 and 27′ in FIGS. 2A-2C may be thought of as extending primarily radially, radial connects or radial connectors herein may have a wide variety of configurations and still be considered a radial connector or radial connect as those phrases are used herein. For example, FIGS. 3A and 3B illustrate radial connectors 37, that may be considered to have a radial dimension as well as a circumferential dimension (which may be measured as an arc around the inflatable wall, or measured linearly) wherein not all of the circumferential dimension is comprised in either the inner or outer member.

When the description herein refers to a fluid “inner volume,” the inner volume may refer to a continuous volume, or it may refer to more than one individual volume that are not in direct fluid communication with each other in the inflatable wall, but together are part of or define an intermediate layer fluid inner volume. For example, in some embodiments, the inflatable wall may comprise an inner fluid volume that has multiple sections, each of which has its own volume that are not in direct fluid communication with each other in the inflatable wall.

In general, intermediate layers herein may be adapted to provide radial support to the inflatable walls when inflated. The intermediate layers herein may impart radial support to the inflatable walls when inflated with a fluid (such as from an external fluid source, which is understood in the art). The intermediate layers may increase the radial and circumferential stiffness of the inflatable wall compared to similar conduits or wall that do not include an intermediate layer. Radial connectors herein may be configured and adapted to impart stiffness to the inflatable chamber. When inflated, radial connects may be put in tension (which may be all radial or distributed radial and circumferential) and the fluid is under compression, and the inflatables herein become significantly stiffer than when uninflated.

The inflatable walls herein may deform less when inflated than when they are not yet fully inflated. When not yet fully inflated, the inflatable walls generally deform more than when fully inflated. And as set forth above, the inflatables are quite flexible and compliant when uninflated, allowing them to collapse easily, such as for delivery to a target location within a patient or subject.

The conduits herein that includes an inflatable wall do not need to include other types of radial supporting components that extend circumferentially around the entire conduit. For example, conduits herein do not need other scaffold sections (e.g., nitinol scaffold patterns) that extend circumferentially around the conduit, as do some other conduits incorporated into other medical devices.

The inner fluid volumes herein (e.g., volume 25, volume 35) generally refer to volumes that are adapted to have their fluid pressure increased upon the delivery of fluid therein. In some embodiments, volumes may be defined by an inner member and an outer member tied or coupled together by one or more optional intermediate or tie layers or members). Terms that may be used instead of volume include “fluid chamber,” “fluid cell,” or other such similar terms that refer to structures that define a volume or space into which fluid may be delivered.

FIGS. 3A and 3B illustrate a sectional end view (the section being transverse to a long axis of the wall when inflated, as shown) of a conduit including an exemplary inflatable wall 30 including an outermost surface 39 and innermost surface 38. Inflatable wall 30 includes intermediate layer 31, which includes plurality of radial connectors 37 disposed between surfaces 38 and 39 as shown, and inner inflatable volume 37. Innermost surface 38 defines inner lumen 36. In this example, the views in FIGS. 3A and 3B may be sectional end views of inflatable wall 40 from FIG. 4. FIGS. 3A and 3B illustrate and are examples of a plurality of radial connectors that each have, in the cross section, a length from a first end that is coupled to the outer layer to a second end that is coupled to the inner layer, as shown. The length in this context refers to the length of the radial connector from first end to second end, and in the cross section as shown. Additionally, the lengths of the plurality of radial connectors are greater than a radial thickness of the outer layer and a radial thickness of the inner layer, the radial thickness measured in a radial direction transverse to the long axis of the inflatable wall, as shown. The radial connectors are extending from and coupled to the inner and outer layers as shown. Any of the radial connectors herein may be unitary with inner and outer members (e.g., 3D printed), or they may be made from different starting materials and bonded together such that they are secured together, both instances of which are examples of being coupled in this context. Unless indicated to the contrary, any of the inflatable walls herein (for example without limitation, wall 40) include radial connectors, wherein the lengths of the plurality of radial connectors (in a cross-section transverse to the wall long axis when inflated) are greater than a radial thickness of an outer layer and a radial thickness of an inner layer, the radial thickness measured in a radial direction transverse to the long axis of the inflatable wall.

FIG. 4 illustrates a perspective view an exemplary conduit including inflatable wall 40 showing some internal components. Wall 40 includes a radially outermost surface and a radially innermost surface as in other examples herein, and an inner lumen as shown. Wall 40 includes an intermediate layer that includes radial connects 47 (only two are labeled), which may be similar to radial connects 37 shown in FIGS. 3A and 3B, and which are disposed in between the radially outermost and radially innermost surfaces. The intermediate layer also includes an inner volume adapted to receive fluid therein to increase the stiffness of the wall 40, which is described in more detail herein. Radial connects 47 at least partially define the inner volume.

In some embodiments the intermediate layer includes inner axial structural members 43′ and outer axial structural members 43″, with inner members 43′ radially inward relative to members 43″. The inner axial members 43′ and outer axial members 43″ can partially define the inner volumes. In some embodiments wall 40 may be made from at least three separate generally cylindrical components, such as inner member, an intermediate member and an outer member. The intermediate member may be disposed within the inner and outer members, and secured to the inner member along axial regions 43″ to form inner axial members 43′. The inner member may be secured to the outer member along axial regions 43″ to form outer axial members 43″.

In some methods of manufacturing, any of the inflatable walls herein that define a lumen may include an inner member (which may comprise one or more layers of material) and an outer member (which may comprise one or more layers of material). The method of manufacturing may include coupling the inner member to the outer member at one or more than discrete locations, which may include point-like locations, linear locations, curved locations, or a combination of linear locations and curved locations. These locations may be referred to herein as secured locations. In these examples, the inflatable wall does not necessarily need to include a separate intermediate member as part of the manufacturing process. In these instances, the one or more radial connectors may be formed by coupling the inner member to the outer member. In some examples the inner and outer members may be spot welded together, for example only.

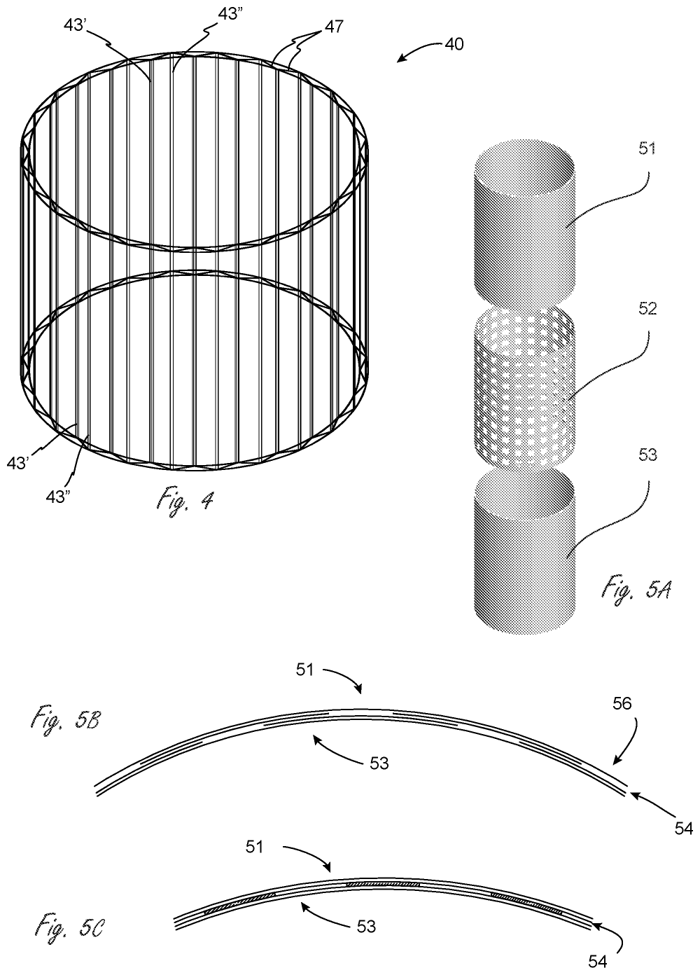

In any suitable example herein, the inflatable wall may be formed from at least two separate components, at least portions of which can be secured to another component to form the inflatable wall. FIGS. 5A-5C illustrates an example of at least three separate components that may be used to form any of the suitable inflatable walls herein. An inflatable wall may be formed from (at least) an outer member 51 (which may be generally cylindrical), an intermediate member 52 or 54 (which may be generally cylindrical), and an inner member 53 (which may be generally cylindrical).

FIGS. 5B and 5C show sectional views of sections orthogonal or transverse to a long axis of the wall when inflated, as are other sectional views herein (e.g., FIGS. 2A-C, FIGS. 3A, 3B, 5B, 5C, 10A, 10B, 15A, 15B, 16C, 17C).

Intermediate members (which in some embodiments may be referred to as tie layers or tie members) herein may be secured to an inner member at one or more secured locations (e.g., secured location 56 shown in FIG. 5B). The secured locations may include one or more discrete locations on an inner surface of the intermediate member and one or more discrete locations on an outer surface of the inner member. The intermediate member may be secured to the outer member at one or more locations. The locations can include one or more discrete locations on an outer surface of the intermediate member and one or more discrete locations on an inner surface of the inner member. Once the inflatable wall is formed, the intermediate member may be part of the intermediate layer of the inflatable wall.

FIG. 5B and FIG. 5C illustrate two different cross sections of the assembled inflatable wall of FIG. 5A. FIG. 5B illustrates a cross section where the tie layer is continuous in the circumferential direction and illustrates how the secured location between inner layer and tie and tie and outer layers shift from side to side of the tie layer. FIG. 5C shows a cross section where the tie layer is discontinuous in the circumferential directions and illustrates how none of the layers are secured to one another at this cross section.

FIG. 6 illustrates an exemplary tie layer or tie member 62, which may be incorporated or used in the manufacture of any suitable inflatable wall herein. FIG. 6 illustrates exemplary locations B, A, wherein the locations A and B that may be secured to (e.g., bonded to) an outer member or outer layer and an inner member or inner layer. The locations A′ and B′ illustrate locations radially opposite the locations A and B which remain unsecured. As shown in FIG. 5A, the wall may include an outer member, an intermediate (or tie) member, and an inner member (there may be additional components as well). Areas or locations “A” are securing (e.g., weld) locations on the exterior of the intermediate member, and may be secured to the outer member. Areas or locations “B” are securing (e.g., weld) locations on the interior to the intermediate member, and may be secured to the inner member. Areas or locations A′ are non-weld locations on the interior of the intermediate member opposite weld areas A on the exterior. Areas B′ are non-weld locations on the exterior of the intermediate tie member that are opposite weld areas B on the interior. The term weld as used herein may be generalized to the term securing, bonding, coupling, or some derivative thereof.

In general, the inflatables herein are in fluid communication with one or more fluid lines or fluid pathways that extend (optionally proximally) towards a fluid source or reservoir (e.g., a fluid). One or more members of the inflatables herein may be are secured together (e.g., at first and second ends of the inflatable) to seal off one or more ends or end regions of inflatable chamber(s) or volumes. For example, at a distal end of the walls herein, an outer member may be secured to an inner member (optionally also secured to an intermediate or tie layer) to create a sealed distal end. Additionally, for example, an outer member may be secured to an inner member (optionally also secured to an inner member, e.g., a tie layer) to create a sealed proximal end of the wall, for example. A fluid tight seal may be created by at least two members secured together (e.g., outer and inner; outer and intermediate; inner and intermediate; outer and intermediate and inner). The two or more layers may additionally or alternatively be secured at any location(s) or continuous location(s) to create any number of separate internal chambers that are not in fluid communication with each other in the inflatable wall.

In some methods of manufacturing the inflatable walls herein, weld areas for some materials like PVC are native, and non-weld areas may be masked with a material which will not weld. Masking may be a metalized area, wherein the weld areas would be regions in which the metallization has been removed. Removal may be chemical etching, or laser ablation, amongst other techniques. Alternatively, weld areas may be treated to enhance weldability of a material that is less susceptible to thermal welding such as by a masked corona treatment, wherein masked areas remain less weldable (as distinct from other methods where the masked portion has better weldability). Coextruded films comprising weld layers such as PET may be used. One such example uses an intermediate tie member of PET which comprises an inner and outer weld layer. The inner and outer surface layers of the inflatable conduit are then comprised of bilayer PET films with the weld layer facing the tie layer. The layers may be laser welded at the appropriate locations. Alternatively, the weld layers can be ablated in the prime areas and welding can be accomplished by pressure/heat welding.

Any of the layers herein may be sheets of material, that have some length, width, and inherent thickness. In some embodiments the structures are fabricated from flat sheets which are assembled and welded into tubular structures. In alternate embodiments the inflatable walls may be fabricated from tubular materials.

In some embodiments the inflatable structures herein are fabricated on an expandable structure (see FIG. 12). In some such embodiments the expandable structure is fabricated from the same or a similar material as the inflatable structure.

Intermediate members herein (e.g., as shown in FIG. 6) may be secured to an inner member and/or the outer member at one or more particular secured locations to impart physical properties to the fluid conduit. Radial connectors herein may be arranged in the intermediate layer to have a regular pattern therein, or they may be arranged in the intermediate layer to have an irregular pattern therein, wherein an irregular pattern may be a result of imparting particular properties to the wall by arranging the radial connectors in the intermediate members in a particular arrangement.

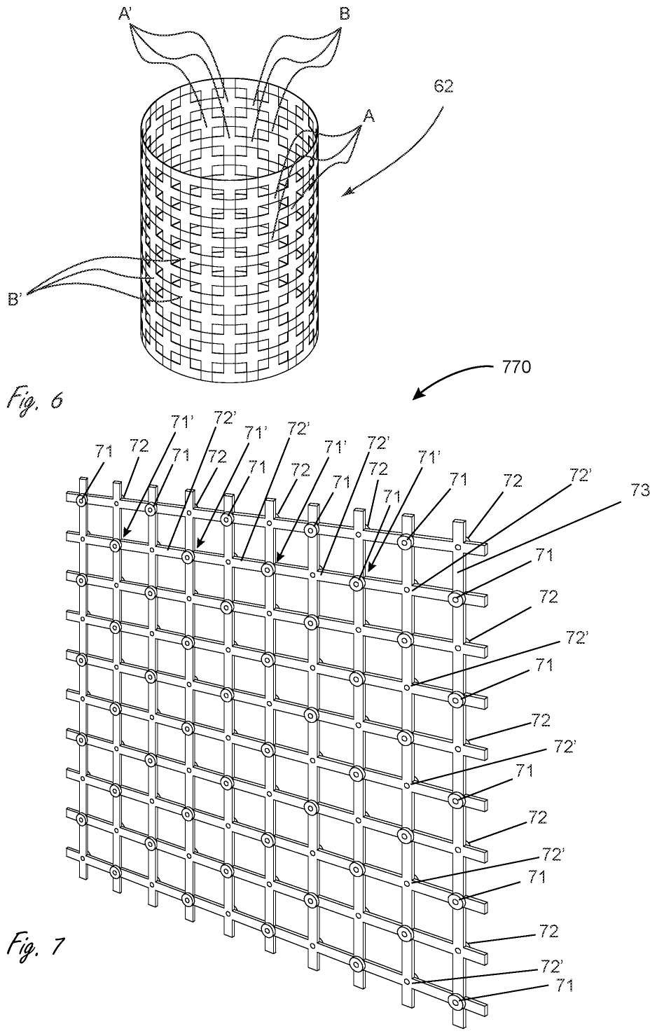

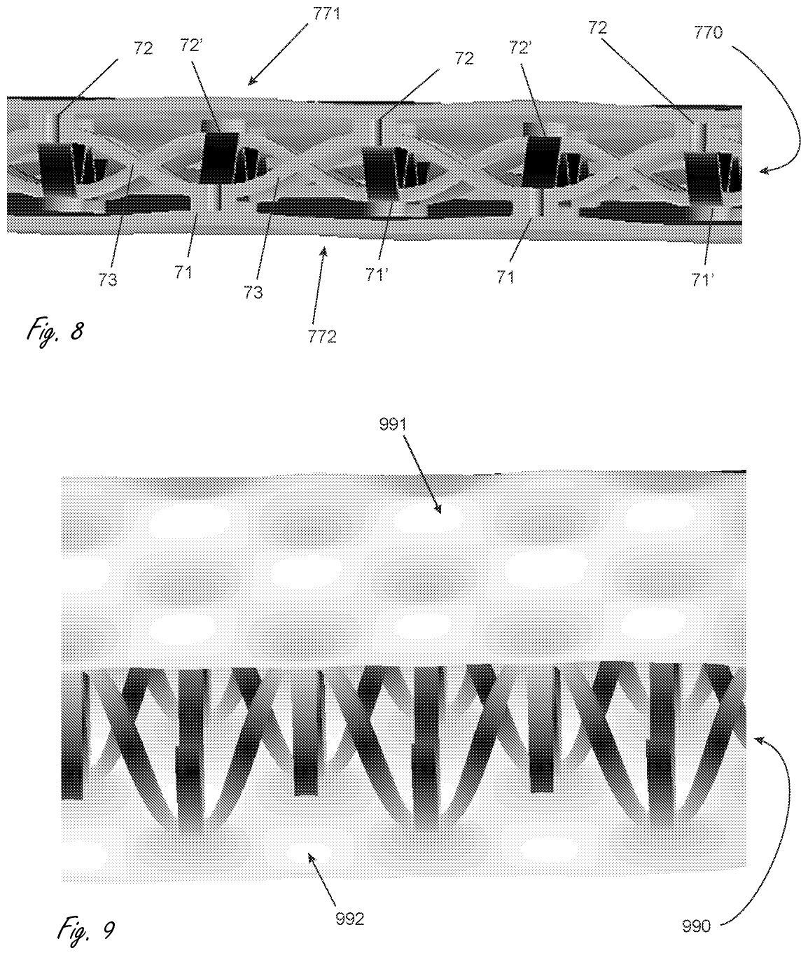

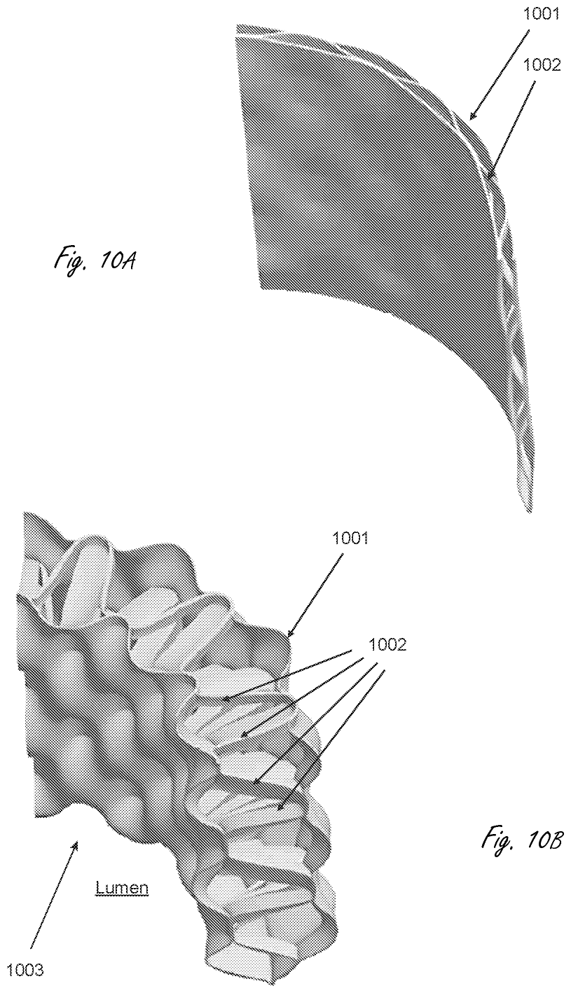

FIG. 7 illustrates an exemplary intermediate member 770 in a flattened configuration that when formed into a cylindrical configuration may be coupled to the inner and outer members, and is described in more detail in the context of the sectional view of FIG. 8. The intermediate member may include one or more securing locations 71, disposed on a first side of the intermediate member, and may have one or more securing locations 72, disposed on a second side of the intermediate member. The securing members 72 may be linearly aligned, as can be seen in FIG. 7. The securing members 72′ may be linearly aligned but in an adjacent “column” or “line” as can be seen in FIG. 7. Securing members 71 can be similarly arranged on the other side of the intermediate member. The securing members may have segments 73 (only one labeled in FIG. 7) in between the securing locations. Locations 71′ and 72′ remain unsecured and sit on the opposite side of the intermediate member from their counterparts 71 and 72 respectively. As shown in FIG. 8, a single row of the intermediate members 72 may be secured to the outer member 771, and the securing members 71 on the other side of the intermediate member 770 are secured to the inner member 722. In a single row, securing members 72 alternate with securing members 71 (e.g., 72-71-72-71-72, etc.), as well as alternative from one side to the other. A plurality of securing members 72 are in an adjacent “column” or “line” to another plurality of securing members 72, and the different pluralities are offset from each other (e.g., in different rows), as shown in FIG. 7 and FIG. 8. These alternating patterns can extend along the entire intermediate member.

The segments 73 shown in FIG. 8 (only two labeled) are considered radial connectors as that term is used herein, and FIGS. 7 and 8 illustrate exemplary methods of forming radial connectors. The radial connectors as shown have first and second ends that are secured to and extend from the inner and outer members, respectively, as shown.

The location(s) at which the intermediate member is secured to the inner and outer members can of course vary from the particular patterns shown in FIGS. 7 and 8, and can be chosen to impart any desired physical characteristics to the inflatable walls herein. The example in FIGS. 7 and 8 is intended to be merely exemplary and not limiting.

Any of the radial connects (radial connectors) herein may have a length and a width, with the width dimension in the axial direction, and the length dimension measured radial/circumferentially (as measured in a cross section that is transverse to a longitudinal or long axis of the inflatable wall when inflated). The radial connectors lengths may be measured from a first end that is coupled to and extending from an inner member to a second end coupled to and extending from an outer member. The width and length of the radial connects may be selected to depending on the desired properties of the inflatable wall, and may be different at different regions of the inflatable to impart desired properties at different locations. Varying the locations of the weld spots can also change the physical properties as desired. For example, it may be beneficial to have stiffer sections in certain areas of the inflatable (e.g., at or near one or more internal components, such as a rotatable impeller for which it may be desirable to maintain a tip gap as is described in the applications incorporated by reference herein), and it may be beneficial to have more flexible sections at certain regions, such as a region axially in between impellers, for example only. In alternate embodiments, some or all radial connects may extend or run at angles other than parallel and perpendicular to the axial direction, such as on a bias. For example, if the intermediate layer from FIG. 7 were rotated 45 degrees (for example without limitation), the radial connects would be not be parallel with and not orthogonal to the long axis of the inflatable wall when inflated.

As used herein, weld is a general term herein and may refer to any type of bond or coupling, including adhesive bonds.

In some methods of manufacturing, an elongate member such as a rod may be advanced in between an outer or inner member and an intermediate member, the elongate member urged to cause the intermediate and outer member to engage, followed by securing the two components together. The same process could be used for securing intermediate and inner members. In examples in which the inflatable wall does not include an intermediate or tie layer, inner and outer members may be bonded directly together, and optionally compressed during at least some portion of the bonding process.

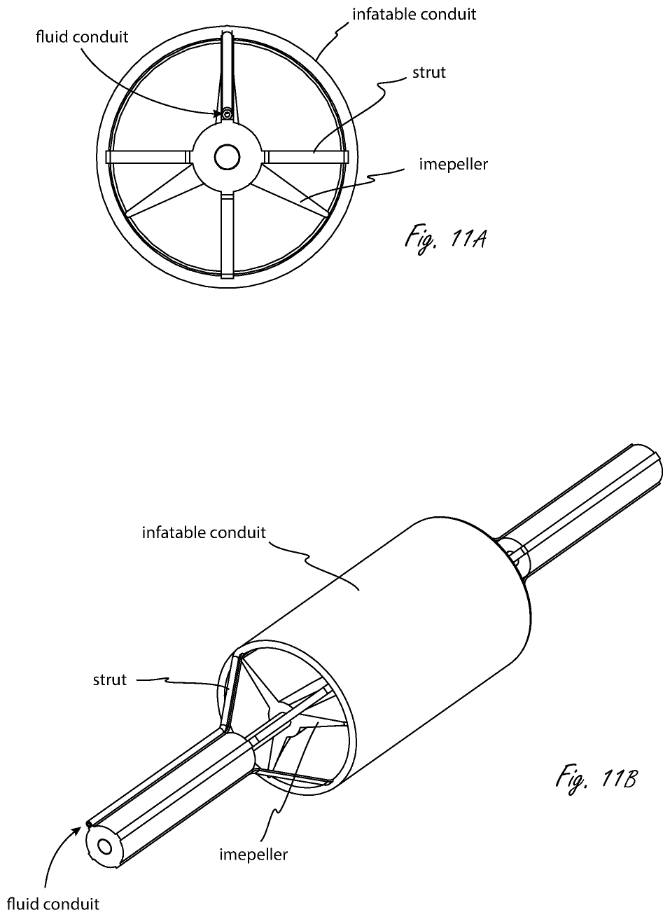

FIG. 9 is another illustration of a portion of an intermediate layer 990 including similar radial connectors to those shown in FIGS. 7 and 8 wherein the securing member comprises just the material associated with bonding the inner 992 or outer 991 layers with the intermediate layer 990.

FIGS. 10A and 10B illustrate additional sectional views (transverse to a long axis) of a portion of an inflatable wall, with radial connectors 1002 configured and arranged in a way that can be the same or similar to those shown in FIGS. 7 and 8. The inflatable wall shown in FIGS. 10A and 10B may be formed with an inner member, and intermediate member, and an outer member, such as is referenced with respect to FIGS. 5-8. Exemplary radially outermost surface 1001 and innermost surface 1003 are also shown.

FIGS. 11A and 11B illustrate a merely exemplary application or implementation of the conduits including an inflatable wall herein. FIGS. 11A and 11B illustrate an exemplary pump portion of an intravascular blood pump. The pump portion includes an expandable and inflatable wall as shown, one or more impellers disposed within the conduit that includes the inflatable wall, and optionally one or more collapsible and expandable struts coupled to the inflatable wall to facilitate collapse of the pump portion. Any and all aspects of any of the blood pumps and pump portions in any of the following references are included herein by reference and may be incorporated into the pump portion in FIGS. 11A and 11B. Additionally, any pump portion and blood pump in any reference incorporated by reference herein may be modified with any of the inflatable walls herein, such as is shown in FIG. 11A and 11B.

FIG. 12 illustrates internal components of the example shown in FIGS. 11A and 11B. FIG. 12 illustrates exemplary struts that may continue along at least a portion of the conduit that includes an inflatable wall, which may help provide radial support to the conduit and/or facilitate collapse of the conduit. The strut extensions may extend through a portion of the inner volume, for example. The strut extensions may alternatively be disposed radially outside of the conduit.

The strut extensions may also be functionally independent of the relationship to the strut. They may be considered structural supports that extend along the length of the conduit, but could be positioned such that they are considered axial extensions of the struts (even if they are separate components).

The optional axial supports shown in FIG. 12 (which are labeled with a term “expandable structure”) may alternatively be inflatable support members, optionally inflated from the same fluid that is fluid communication with the inflatable wall such that it can be used to inflate the inflatable wall.

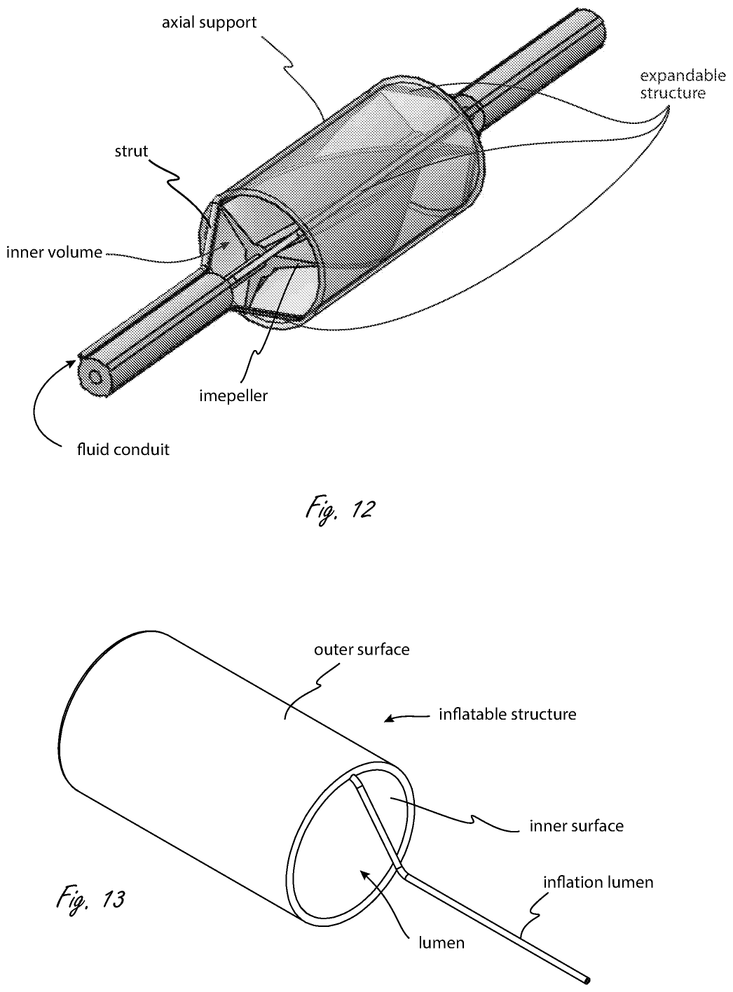

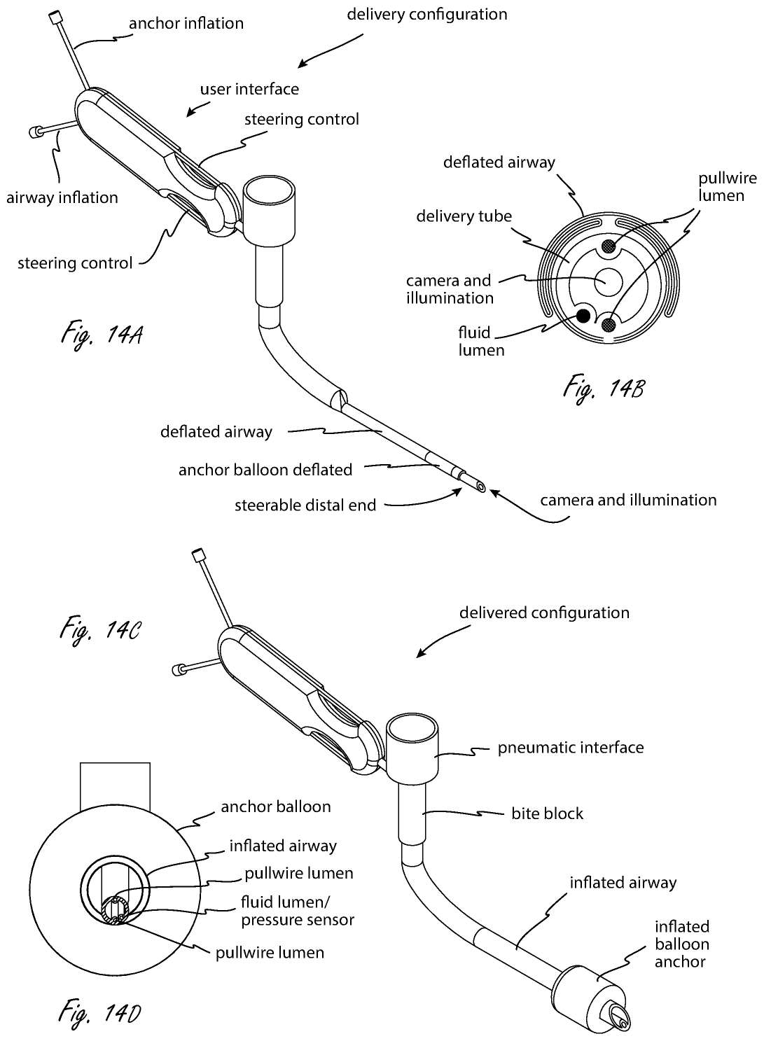

As is set forth above, the inflatable concepts herein may be used in a variety of applications. FIG. 13 illustrates an exemplary application of an inflatable that comprises an inflation tube and an inflatable wall. The inflatable wall as illustrated can comprise any of the configurations and features of any other fluid conduit described herein. The inflatable conduit such as any of those herein comprise an inner lumen defined by an inner surface, and an outer surface. Such a device may be used to maintain patency including but not limited to body lumens such as arteries and veins, gastrointestinal lumens, bile and other body ducts, airway lumens such as trachea and bronchi. In some embodiments of such inflatable conduits the inflation lumen is filled with a fluid such as saline, in others it may be filled with a gas such as CO2. In others associated with permanent implants, the conduit may be filed with a material which attains a solid state after delivery. Some such solidifying components include, but are not limited to, epoxies and hydrogels.

FIGS. 14A-14D illustrate another exemplary application of the inflatable concepts herein. FIGS. 14A-D illustrate an expandable endotracheal device comprising an inflatable conduit, which may include any of the features of any of the inflatable walls described herein. The endotracheal device comprises a number of useful features, examples of which are described as follows. The distal portion of the airway flow tube comprises inflatable wall collapsed around a delivery tube during delivery and inflated after delivery. It may again be deflated for repositioning or removal. This allows for a smaller less traumatic cross section during delivery for that portion of the system which passes through the pharynx and larynx. The endotracheal device comprising a proximal interface (configured for connection to a ventilator or ventilation bag. The proximal portion of the airway flow tube comprises a rigid bite plate (stationary or movable). A distal portion comprises an inflatable tracheal anchor distinct from or comprised in the inflatable airway. Some embodiments may include one or more imaging sensors located on a distal portion of the airflow tube. The imaging sensors may be located within the inflatable anchor. Alternatively, or in combination, the imaging sensors can be located within a distal portion of the air flow conduit delivery tube. In some embodiments the imaging sensors comprise an illumination source, in others an illumination source is located separately, such as an illumination source in the anchor structure and an imaging sensor located within a distal portion of the air flow conduit delivery tube.

In some embodiments the air flow tube is steerable from the proximal end when in the delivery configuration. In the embodiment illustrated the delivery tube is steerable via two steering wires (not shown) which travel within the steering wire lumens and are affixed at the proximal ends of the steering controls and at the distal end of the delivery conduit. Compression of a steering control tensions the controls steering wire thereby causing the distal end of the tracheal tube to bend in the direction of the steering control. In some embodiments there may only be one control thereby limiting steering to on direction.

FIGS. 14A and 14B (sectional) illustrate an inflatable endotracheal tube in a delivery configuration and FIGS. 14C and 14D in a delivered inflated configuration. FIG. 14B illustrates a distal end view of the distal end of the inflatable endotracheal tube. FIG. 14D illustrates a cross section of the delivered endotracheal tube through the tracheal anchor.

Any of the inflatables herein may be in fluid communication with one or more fluid sources, such as external fluid reservoirs disposed outside of a subject, the basic concept of which is understood by those skilled in the art. The fluid communication may communicate one or more fluid pathways, which may include one or more fluid lines or fluid lumens, such as any fluid pathways or lines described in any of the applications incorporated by reference herein.

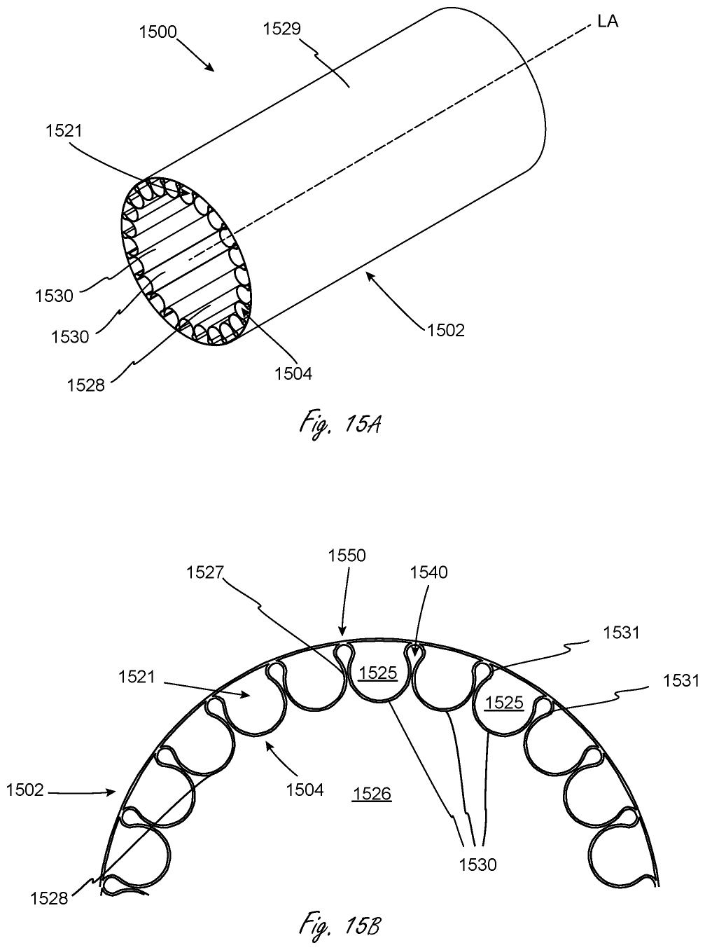

FIGS. 15A-17C illustrate alternative conduits with an inflatable wall and exemplary methods of manufacturing the inflatables. Any of the features of any of the inflatables or inflatable walls described or claimed herein may be incorporated into the inflatable walls described with reference to FIGS. 15A-17C, and vice versa. FIGS. 15A-17C illustrate alternative inflatable conduits that include an intermediate layer disposed radially between an innermost surface of the inflatable conduit that at least partially defines the lumen and a radially outermost surface of the inflatable conduit. The intermediate layer in FIGS. 15A-17C includes one or more radial connectors in between the innermost and outermost surfaces of the inflatable conduit, and a volume adapted to be put into fluid communication with a fluid source to inflate the volume.

FIG. 15A illustrates a perspective sectional view of a conduit including an inflatable wall 1500 in an inflated configuration, the section transverse to the long axis LA as shown (a long axis (“A”) is also shown in FIG. 2A). FIG. 15B illustrates a partial end view of inflatable wall 1500. Wall 1500 includes an outermost surface 1529 and an innermost surface 1528 defining lumen 1526 (see FIG. 15B). Wall 1500 also includes intermediate layer 1521 (see FIG. 15B), which in this embodiment includes a plurality of radial connects 1527 and inner volume 1525, which in this embodiment are a plurality of individual, axially extending fluid pathways as shown.

In this exemplary embodiment inner layer 1504 has or defines a corrugated configuration when the wall 1500 is inflated, as shown. The corrugated configuration of inner member 1504 includes a plurality of ridges 1530 (only a subset are labeled for clarity) and a plurality of grooves 1531 circumferentially in between the ridges (only a subset of grooves are labeled for clarity).

Inflatable wall 1500 in this embodiment also includes outer member or layer 1502 secured to inner member or layer 1504 at a plurality of secured or coupling locations or regions 1550 (FIG. 15B) that are circumferentially spaced apart around the wall as shown, and which in this example are elongate and generally axially extending secured locations along the length of the conduit 1500, as shown in FIG. 15A. The coupling between the inner layer and outer layer at regions 1550 is at the locations of grooves 1531 of the inner member 1504. Coupling the inner member 1504 and outer member 1502 at axially extending secured or coupling regions 1550 (and not at locations circumferentially in between secured regions 1550) creates a plurality of elongate axially extending inflatable volumes 1525 (only two are labeled for clarity) that in this embodiment are parallel with the long axis LA, as shown in FIG. 15A.

In this embodiment, in the transverse cross section shown in FIGS. 15A and 15B, the inner layer 1504 has an inner length between the circumferentially spaced secured locations that is greater than the length of outer layer 1502 between the respective circumferentially spaced secured locations, as shown. The relatively longer length of the inner member causes the inner member, in between the secured locations 1550, to expand toward the configuration shown in FIGS. 15A and 15B, such that each of the circumferentially adjacent fluid pathways that define volumes 1525 is configured to interfere with at least one adjacent fluid pathway when inflated with a fluid to thereby stiffen the inflatable wall, as can be seen in FIG. 15A.

Inflatable volumes 1525 may be in fluid communication with one or more fluid sources or reservoirs, and the delivery of fluid into volumes 1525 inflates volumes 1525 to increase pressure therein and therefore stiffening wall 1500.

Inner layer 1504 is not bonded to itself at interfering locations 1540, but those regions of the inner member 1540 physically engage and interfere with one another when fluid is delivered through the fluid pathway in volumes 1525, as can be seen in FIG. 15A. The interaction at interfering locations 1540 when inflated prevent the conduit from collapsing and help increase the stiffness of the inflatable wall.

Inner layer 1504 in this embodiment may also be described as having a wavy configuration when the wall is inflated, wherein the inner member includes a plurality of peaks and valleys as shown. The plurality of peaks are shown extending or facing radially inward toward a long axis (not labeled but which is described elsewhere herein).

In any of the embodiments herein, any of the individual volumes 1525 (FIG. 15B) may be in fluid communication with one or more different fluid sources. For example, some volumes may in fluid communication with a first fluid source and a second subset of the volumes may be in fluid communication with a second fluid source. Additionally, in methods of use, some of the volumes in fluid communication with a first fluid source may be inflated with a first fluid while other volumes do not necessarily need to be inflated.

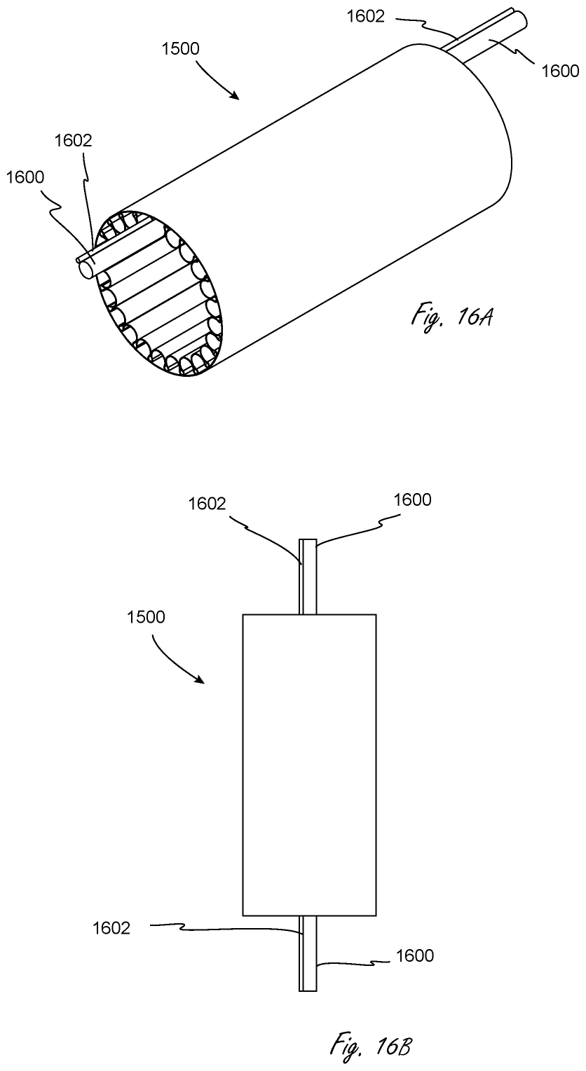

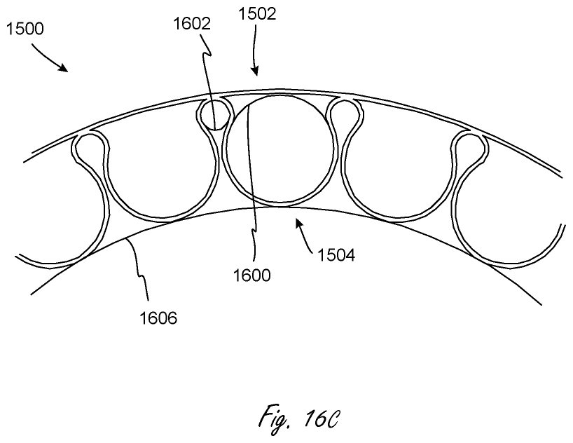

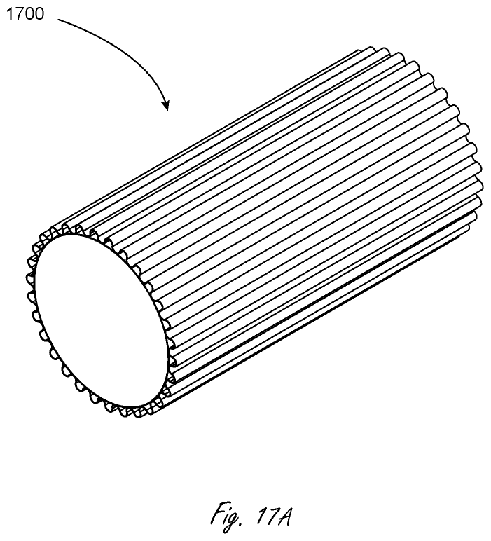

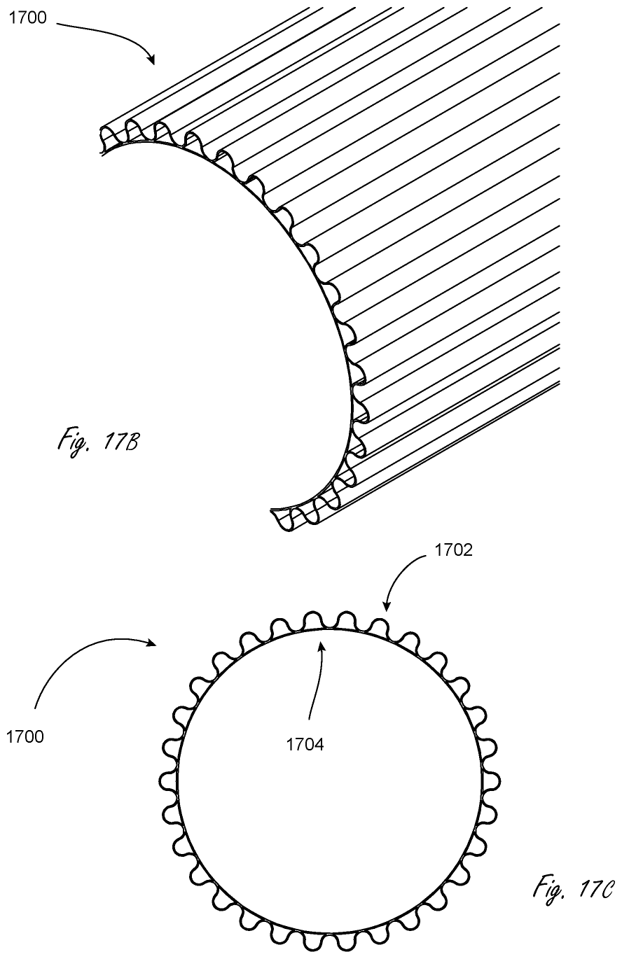

FIGS. 16A-16C illustrate a merely exemplary method of manufacturing the conduit with the inflatable wall shown in FIGS. 15A-15C. Any of the wall reference numbers are considered to be applied to FIGS. 16A-16C even if the figures do not expressly include the reference number (e.g., wall 1500).

An exemplary method of manufacturing inflatable wall 1500 includes providing an inner mandrel 1606. A first plurality of cylindrical members 1600, with a first diameter, can be placed around the inner mandrel extending longitudinally as shown. Only one cylindrical member 1600 is shown, but any number of cylindrical members 1600 may be placed about the inner mandrel 1606. A plurality of second cylindrical members 1602, with smaller relative diameters, may be positioned in between the first plurality of cylindrical members 1600 such that long axes of the second cylindrical members 1602 are further radially outward compared to long axes of the first plurality of cylindrical members 1600, as shown. Inner member 1504 may then be woven about the plurality of members 1504 and 1502, in a manner in which the inner member is woven radially inward relative to one of the plurality of cylindrical members 1600, then radially outward of one of the adjacent second cylindrical members 1602, continuing radially inward of an adjacent one of the plurality of cylindrical members 1600, and continuing in this manner around the members 1600 and 1602.

An outer member 1502 may then be placed over or about the inner member 1504, which has a corrugated configuration after being woven through members 1600 and 1602. The outer member 1502 and inner member 1504 may then be bonded at locations 1550 (see FIG. 15B), and the cylindrical members 1600 and 1602 may then be removed, leaving the bonded inner and outer members.

It is understood that other methods of manufacturing the conduits herein may be employed, and the disclosure relative to FIGS. 16A-16C is only exemplary.

In some examples, the inner and outer members 1504 and 1502 may be sheets of material with a first side or face comprising a first material and a second side or face comprising a second material. The materials selected may help bond or secure the inner and outer layers together. For example only, the inner and outer layers may comprise nylon impregnated with thermoplastic polyurethane (TPU) in a portion of the layer. As an example, the first and second layers may each comprise (TPU) on a first surface wherein a second surface does not comprise TPU. The TPU sides of the two sheets may be secured to each other at locations 1550. In any of these examples the bonding may occur with heat shrinking. Nylon, or a similar material, may help reinforce the first and second layers, and it or a similar material will not adhere to other surface when heat shrunk, optionally onto a mandrel. In alternative embodiments, the first and second layers may include TPU throughout the layer as opposed to only in a portion of the layer. In alternative embodiments, the first and second layers may comprise PEBAX® in a portion thereof or throughout the layer.

In embodiments in which the layers include a material such as TPU or similar material, the method of manufacturing includes securing the inner and outer members at regions 1550, but not at regions circumferentially in between the securing regions. The approach described with respect to FIGS. 16A-16C including members 1600 prevents portions of inner layer 1504 from contacting and bonding to outer 1502. For example, members 1600 maintain inner layer 1504 away from outer layer 1502 wherein the members 1600 are disposed. Additionally, relatively smaller diameter members 1602 can urge axial sections of the inner layer 1504 radially outward to ensure they are in contact with outer layer 1502 when bonding the two layers at regions 1550.

In some alternative methods of manufacture, the mandrel and/or members 1600 and 1602 may comprise a dissolvable mandrel, such as a polyvinyl alcohol. After the inner and outer layers are bonded together, the mandrels may be dissolved away, leaving the formed conduit.

In alternative methods of manufacturing, the conduits may be printed, including printing intermediate radial connects at desired locations, which may include any particular location as described herein for all purposes. For example, the conduits may be printed silicone elastomer with the desired configuration. The conduits may be printed in the inflated and expanded configuration.

In any of the embodiments herein, the conduits may optionally be collapsible to a size as little as 13 F (or less), and may be inflatable and expandable to a size as great as 40 F. In any of the embodiments herein the inflated configuration may be 2-5 times an uninflated and collapsed configuration. In some embodiments the inner diameter of the conduit is 12-15 mm when inflated and expanded, and wherein the conduit is adapted to be collapsible to have an outer dimension (e.g., diameter) of 3-6 mm.

FIGS. 17A-17C illustrate an exemplary inflatable conduit that may incorporate any feature from any other embodiment herein, and vice versa. For example, exemplary inner member 1704 and outer member 1702 are shown. Inflatable wall 1700 in FIGS. 17A-17C may be substantially similar to conduit 1600 shown in FIGS. 16A-16C (and manufactured similarly), except the inner and outer layers are reversed. Inflatable wall 1700 includes an outer member with a wavy or corrugated configuration as shown. All other features shown and described with respect to wall 1600 may be similarly incorporated into wall 1700 and the description thereof with the appropriate modification thereto.

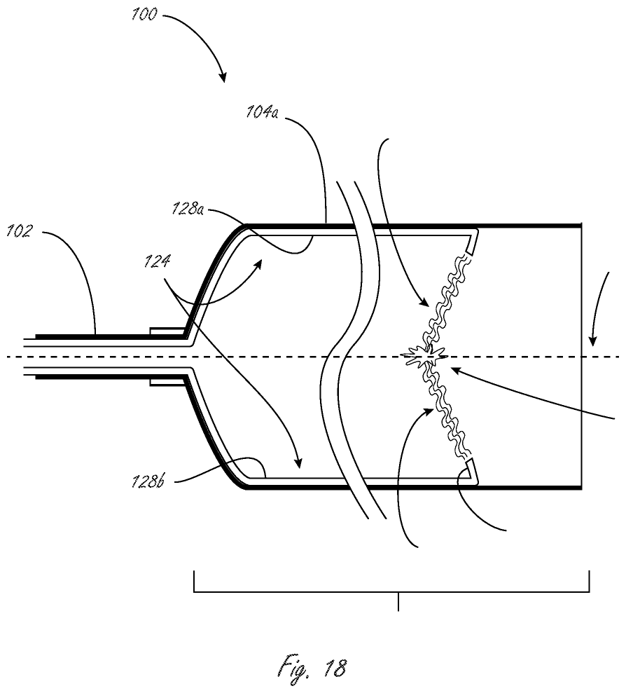

In some exemplary applications, the inflatable concepts herein may be incorporated into devices and systems that are adapted for removing a thrombus from a patient's blood vessel. FIG. 18 illustrates a merely exemplary thrombus removal system 100 in which one or more components may incorporate one or more inflatable features herein. Elongated catheter 102 of the system 100 may be positioned within a patient's pulmonary vasculature. Elongated catheter 102 includes distal portion 104a as shown. System 100 may be adapted to capture, macerate and/or fragment a thrombus using irrigation alone, which may incorporate utilizing a fluid delivery pathway described above with respect to the embodiments shown in any of FIGS. 15A-17C, which is described in more detail below. In the example of FIG. 18, openings 139a-b of fluid delivery mechanism 124 are positioned, oriented and arranged to direct fluid jets 136a-b to intersect with one another at fluid intersection 137. In some embodiments, openings 139a-b are optionally arranged such that fluid jets are directed at least partially proximally (e.g., backward) as shown in FIG. 18. In some embodiments, the openings 139a-b are arranged such that fluid jets are directed at an angle that is one or more of normal (or substantially normal) to a central axis 141 of the chamber 109 or at least partially distally (e.g., toward distal opening). While intersection 137 is depicted near a central region of expandable chamber 109, it will be appreciated that the openings 139a-b may be positioned, oriented and/or or disposed such that the intersection of the fluid jets may be in a variety of locations within the chamber 109. Forces generated by the fluid jets 136a-b at the intersection 137 may one or more of macerate/fragment a thrombus or urge the thrombus fragment(s) to be evacuated proximally along catheter 102.

In some implementations of the embodiment shown in FIG. 18, distal portion 104a as shown may be any of the inflatable walls herein, and in fact all of catheter 102 may be an inflatable wall and include any of the features of any of the inflatable walls herein (e.g., FIGS. 1-17C). In some particular examples, elongate fluid delivery tubes 128a and 128b may be replaced by the fluid pathways that each define one or more volumes 1525 (e.g., FIG. 15B), which may be coupled to or part of the distal portion 104a, which in this embodiment includes the funnel shape as shown in FIG. 18. In these examples, fluid delivery pathways 128a and 128b (or any number of additional pathways) may have open ends (i.e., not sealed) that create the distal ports or openings 139 from which the fluid jet is delivered, as shown.

The distal portion 104a may be configured as is inflatable wall 1500 in FIGS. 15A-C, and one of the fluid delivery lumens may be configured to extend radially inward relative to the remainder of the conduit such that the distal opening is facing generally radially inward as shown in FIG. 18. Fluid pathways 128a/b may be in fluid communication with a first fluid source and the remainder of the inflatable conduit may be in fluid communication with a second fluid source, for example, such that fluid pathways 128a/b are adapted and positioned to deliver the fluid jets, while the remainder of the intermediate layer of the conduit 1500 may be adapted to receive inflatable fluid therein and in response thereto to increase the stiffness of the conduit. In some embodiments of FIG. 18, the fluid pathways that supply the jetting fluid may be stiff, that is, they may be configured to hold or maintain their shape without requiring an inflation fluid to be delivered therein.

1. An intravascular inflatable medical device, comprising:
a conduit including an inflatable wall, the inflatable wall defining a lumen therein,
the inflatable wall including an outer layer, an inner layer, and an intermediate layer that is disposed radially between the outer layer and the inner layer, the inner layer at least partially defining the lumen,
the intermediate layer including, in a cross-section transverse to a longitudinal axis of the inflatable wall when inflated, an inflatable volume and a plurality of radial connectors extending between the outer layer and the inner layer, such that when a fluid is delivered into the inflatable volume, a stiffness of the inflatable wall increases.

2. The medical device of claim 1, wherein the inflatable wall is compliant when uninflated such that it is adapted to be folded for delivery, wherein when folded it can have a different general configuration than when inflated.

3. The medical device of claim 1, wherein the plurality of radial connectors are arranged so as to have a regular pattern along a length of the intermediate layer.

4. The medical device of claim 1, wherein the plurality of radial connectors are arranged so as to have an irregular pattern along a length of the intermediate layer.

5. The medical device of claim 1, wherein the plurality of radial connectors are arranged within the intermediate layer such that a first portion of the inflatable wall has a first stiffness and a second portion spaced from the first portion has a second stiffness that is different than the first stiffness when the inflatable wall is inflated with the fluid.

6. The medical device of claim 1, further comprising a fluid pathway extending proximally from the inflatable wall and in fluid communication therewith to facilitate fluid delivery through the fluid pathway and into the inflatable wall.

7. The medical device of claim 1, wherein each of the radial connectors extends from the outer layer at a location that is one or more of axially or circumferentially spaced from a location from which the respective radial connector extends from the inner member.

8. The medical device of claim 7, wherein the plurality of radial connectors together create a wave pattern.

9. The medical device of claim 1, wherein the inflatable volume comprises a first inflatable volume and a second inflatable volume, wherein the first and second inflatable volumes are not in direct fluid communication within the inflatable wall.

10. The medical device of claim 9, wherein the first and second inflatable volumes are in fluid communication with first and second fluid inflation pathways, respectively.

11. An intravascular inflatable medical device, comprising:
a conduit including an inflatable wall, the inflatable wall defining a lumen therein,
the inflatable wall including an outer layer, an inner layer, and an intermediate layer that is disposed radially between the outer layer and the inner layer, the inner layer at least partially defining the lumen,
the intermediate layer including, in a cross-section transverse to a longitudinal axis of the inflatable wall when inflated, an inflatable volume and a plurality of radial connectors extending between the outer layer and the inner layer,
wherein the plurality of radial connectors each have a length from a first end that is coupled to the outer layer to a second end that is coupled to the inner layer,
wherein the lengths of the plurality of radial connectors are greater than a radial thickness of the outer layer and a radial thickness of the inner layer, the radial thickness measured in a radial direction transverse to the longitudinal axis of the inflatable wall.

12. The medical device of claim 11, wherein the inflatable wall is compliant when uninflated such that it is adapted to be folded for delivery, wherein when folded it can have a different general configuration than when inflated.

13. The medical device of claim 11, wherein the plurality of radial connectors are arranged so as to have regular pattern along a length of the intermediate layer.

14. The medical device of claim 11, wherein the plurality of radial connectors are arranged so as to an irregular pattern along a length of the intermediate layer.

15. The medical device of claim 11, wherein the plurality of radial connectors are arranged within the intermediate layer such that a first portion of the inflatable wall has a first stiffness and a second portion spaced from the first portion has a second stiffness that is different than the first stiffness when the inflatable wall is inflated with the fluid.

16. The medical device of claim 11, further comprising a fluid pathway extending proximally from the inflatable wall and in fluid communication therewith to facilitate fluid delivery through the fluid pathway and into the inflatable wall.

17. The medical device of claim 11, wherein each of the radial connectors extends from the outer layer at a location that is one or more of axially or circumferentially spaced from a location from which the respective radial connector extends from the inner member.

18. The medical device of claim 17, wherein the plurality of radial connectors together create a wave pattern.

19. The medical device of claim 11, wherein the inflatable volume comprises a first inflatable volume and a second inflatable volume, wherein the first and second inflatable volumes are not in direct fluid communication within the inflatable wall.

20. The medical device of claim 19, wherein the first and second inflatable volumes are in fluid communication with first and second fluid inflation pathways, respectively.

21-29. (canceled)
